(19) United States
(12) Patent Application Publication
(10) Pub. No.: US 2008/0082070 A1
(43) Pub. Date: Apr. 3, 2008

(54) Method of marketing incontinence products

(75) Inventors: David Arthur Fell, Neenah, WI (US); Kenny Lee Johnson, Neenah, WI (US); Stephen Bradford Cook, Appleton, WI (US); Toan Thanh LeMinh, Neenah, WI (US); Brian Lee Thomas, Appleton, WI (US); Jerome Steven Veith, Menasha, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/540,233

(22) Filed: Sep. 29, 2006

(51) Int. Cl. A61F 13/15

(52) U.S. Cl. 604/385.02; 702/182

(57) ABSTRACT

A method of marketing incontinence products is disclosed. The method includes creating a product offering of incontinence products. The product offering includes first, second and third segments that are distinguished from one another by display segmentation elements. The first segment is directed to male incontinence consumers, the second segment is directed to female incontinence consumers, and the third segment is directed to caregivers. The product offering is then communicated to a retailer and a product display is created at a retail site that resembles the product offering. Either simultaneously or sequentially, the product offering is communicated to potential purchasers, using multimedia advertisement, to inform them of the availability of such disposable absorbent incontinence products.

### BACKGROUND OF THE INVENTION

Today, a large variety of disposable absorbent incontinence products are offered for sale at many retailers. By “disposable absorbent incontinence products” it is meant pull-on type underwear, adjustable underwear, refastenable underwear, various styles of undergarments, fitted briefs, guards for men, pads, pantiliners, etc. Some products are directed to male users while others are directed to female users, and still others to caregivers. Most products are available in a plurality of sizes to fit the particular rise and waist circumference of a consumer. By “rise” it is meant the longitudinal distance of a product starting from the front edge located adjacent to the waist opening and extending through the crotch region to the back edge located adjacent to the waist opening. By varying the rise of a product, it can better accommodate the anatomical shape of certain consumers. By “waist circumference” it is meant the circumferential distance around the waist opening. For example, some products are sized for consumers having a small waist, other products are sized for consumers having a regular-size waist, still other products are sized for consumers having a large waist, and still other products are sized for heavy set consumers having an extra large waist. Some products are constructed to offer a variety of absorbent capacities so as to accommodate a person's fluid discharge pattern and flow rate.

Disposable absorbent incontinence products are also offered which have an open configuration, such as a fitted brief, which need to be attached about the wearer's torso using tapes or a belt. Other disposable absorbent incontinence products are offered which have a unitary configuration, which simulates regular cloth underwear and are designed to have the consumer step into them before they are pulled up around their torso. Furthermore, some disposable absorbent incontinence products are offered either scented or unscented to satisfy the particular preference of a consumer.

Consumers of incontinence products benefit from having such a large selection of products from which to choose to meet their individual protection needs. However, the plethora of product offerings makes product selection difficult for the shopper and makes product display difficult for the retailer. One such difficulty is that when a consumer shops at different retail establishments, he or she often finds that it is difficult to quickly locate and select the specific product that they desire. Sometimes, their preferred product is not carried by a particular retailer. This is because each retailer decides which products to carry and how such products are to be arranged and displayed on their store shelves. Obviously, a small retailer does not have the same amount of shelf space available as a large retailer to display merchandise.

Another challenge for manufacturers of disposable absorbent incontinence products is that they commonly advertise their products on television, in newspapers, in flyers inserted into newspapers, on web sites, as well as by other means wherein a graphical image of the product package is conveyed to the potential purchaser. However, when that purchaser shops at their local store, they may not find a product package that corresponds to the graphical image shown in the advertisement. Sometimes this occurs because the most recently introduced product has not yet made it to the store shelves or because that particular store does not stock the new product.

Now a method of marketing disposable absorbent incontinence products has been invented which addresses these issues.

### SUMMARY OF THE INVENTION

Briefly, this invention relates to a method of marketing incontinence products, especially disposable absorbent incontinence products. The method includes creating a product offering of incontinence products. The product offering includes first, second and third segments that are distinguished from one another by display segmentation elements. The first segment is directed to male incontinence consumers, the second segment is directed to female incontinence consumers, and the third segment is directed to caregivers. The product offering is then communicated to a retailer and a product display is created at a retail site that resembles the product offering. Either simultaneously or sequentially, the product offering is communicated to potential purchasers, using multimedia advertisements, to inform them of the availability of such disposable absorbent incontinence products.

### BRIEF DESCRIPTION OF THE DRAWINGS

FIG. 1 is a flow diagram of a method of marketing incontinence products.

FIG. 2 is a schematic of first, second and third segments, each distinguished from one another by display segmentation elements.

FIG. 3 is another flow diagram of a method of marketing incontinence products.

FIG. 4 is still another flow diagram of a method of marketing incontinence products.

### DETAILED DESCRIPTION

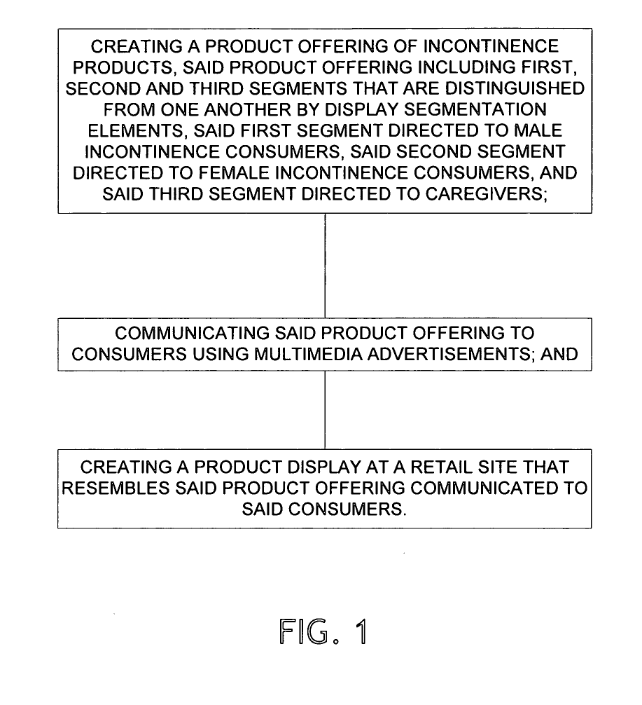

Referring to FIG. 1, a flow diagram is shown depicting a method of marketing incontinence products. An “incontinence product” is a product that is designed to be worn by a person who suffers from incontinence. By “incontinence” it is meant that the person is incapable of completely controlling his or her excretory functions. Most people who suffer from incontinence can not restrain or control the discharge of human exudate, such as body fluids, especially urine, and/or fecal matter from their bowels. Urinary incontinence is the most prevalent form of incontinence in adults. However, some people do suffer fecal incontinence wherein they can not control the release of feces from their bowels.

Incontinence products can be worn by people of all ages, including infants, toddlers, teens and adults. However, most incontinence people are adults over the age of forty. Some people are not incontinent but have limited mobility or are bedridden and need to use incontinence products. For example, some bedridden people who live at home, in a hospital or in a nursing home, desire or require an incontinence product. Other people who may suffer from an illness or infirmity require an incontinence product.

Many incontinence products are designed to be worn around a person's torso, similar to regular cloth underwear. Disposable absorbent incontinence products are available that can absorb and retain discharged urine and other body waste while the wearer is active, mobile, semi-immobile or immobile. A semi-immobile person is able to move about to a limited extent. For example, a semi-immobile person may be able to move about a room or short distances on their own or with the assistance of a wheelchair or walker. An immobile person is one who is bedridden or is confined to a chair or bed and can not physically move without the assistance of at least one other person.

Disposable absorbent incontinence products are designed to be removed and discarded after a single use. By “single use” it is meant that the disposable absorbent incontinence product will be disposed of after being used once instead of being laundered or cleaned for reuse, as is typical of regular cloth underwear. Examples of some commercially available disposable absorbent incontinence products include pads, pantiliners, fitted briefs, belted shields, guards for men, protective underwear and adjustable underwear. All of the above products are commercially sold under the DEPEND or POISE brand names. DEPEND and POISE are registered trademarks of Kimberly-Clark Corporation, having an office at 401 North Lake Street, Neenah, Wis. 54956.

Many of the disposable absorbent incontinence underwear are similar in appearance, size and shape to regular cloth underwear except that they are formed from a variety of different materials including absorbent and elastic materials. The absorbent materials allow the disposable absorbent incontinence underwear to absorb and retain body waste while the elastic material permits the disposable absorbent incontinence underwear to snugly conform to the anatomy of the wearer's torso.

Still referring to FIG. 1, the inventive method of marketing incontinence products includes creating a product offering which includes first, second and third segments that are distinguished from one another by display segmentation elements. The first segment is directed primarily to male incontinence customers, shoppers, choosers and users, herein collectively referred to as male incontinence consumers. The male incontinence consumers will be interested in various disposable absorbent incontinence products, including fitted briefs, guards for men, protective underwear and adjustable underwear. The first segment should include at least two distinct incontinence products bearing indicia denoting targeted applicability to male consumers. Some of the male incontinence products will have an absorbent core that is located forward toward the front edge of the incontinence product to better accept body fluid discharged from a male user. The various sizes of the disposable absorbent male incontinence products can have different absorbent capacities. However, for male underwear, it is possible to design and manufacture them with a single absorbent capacity or with two or more different absorbent capacities. By manufacturing the male incontinence underwear with a single absorbent capacity, a manufacturer can reduce the cost of manufacturing the various sizes of underwear. For example, all the male incontinence underwear could utilize the same absorbent core.

It should be noted that male incontinence underwear is a totally different product from fitted briefs and belted shields, and one skilled in the art would not expect all of these products to have the same absorbent capacity as male incontinence underwear.

Disposable absorbent male incontinence underwear is available in a plurality of sizes to fit the particular rise and waist circumference of a consumer. By “rise” it is meant the longitudinal distance of a product starting from the front edge located adjacent to the waist opening and extending through the crotch region to the back edge located adjacent to the waist opening. By varying the rise of a product, it can better accommodate the range of anatomical shapes of certain consumers. By “waist circumference” it is meant the circumferential distance around the waist opening. Male incontinence underwear is normally designed to have a specific range of rise and waist opening dimensions to comfortably fit the male consumer.

Much of the disposable absorbent male incontinence underwear sold today has a unitary configuration which is similar to regular cloth underwear. By “unitary configuration” it is meant that the disposable absorbent male incontinence underwear is constructed with a waist opening and a pair of leg openings and needs to be pulled onto the body like normal underwear. The unitary configuration is designed to have the wearer step into the waist opening and insert a foot through each of the leg openings. The unitary disposable male incontinence underwear is then pulled up along the wearer's legs and thighs until it reaches the wearer's torso. Once positioned on the wearer's torso, the disposable male incontinence underwear can be adjusted to comfortably cover the abdomen, buttocks and crotch region of the male user. Two examples of unitary underwear are DEPEND Extra Underwear and DEPEND Super Plus Underwear codes commercially sold by Kimberly-Clark Corporation. DEPEND is a registered trademark of Kimberly-Clark Corporation, having an office at 401 North Lake Street, Neenah, Wis. 54956. Another embodiment of a unitary disposable absorbent underwear is disclosed in published U.S. patent application Ser. No. 2004/0210205 A1 to Van Gompel et al.

Other disposable absorbent incontinence underwear has an open configuration. By an “open configuration” it is meant that the disposable absorbent incontinence underwear does not have a waist opening and a pair of leg openings before it is positioned about the wearer's torso. Typically, disposable absorbent incontinence underwear having an open configuration has a relatively flat or convex shape before it is secured around the torso of the wearer. Commonly, disposable absorbent incontinence underwear having an open configuration has an approximately rectangular or hourglass shape. A fitted brief is a good example of an open configuration product since it has a large rectangular shape and is designed to be positioned about the user's crotch. The fitted brief extends upwards in the front and back to cover the abdomen and the buttock. Two commercially available fitted briefs sold today are the Walgreen's Disposable Brief and the DEPEND Maximum Protection brief. Fitted briefs are also disclosed in U.S. Pat. No. 4,500,316 issued to Damico.

Disposable absorbent incontinence underwear which has an open configuration is normally designed to be placed on the wearer's body proximate the wearer's torso. Disposable absorbent incontinence underwear having an open configuration can have a front region designed to cover the wearer's abdomen, a crotch region designed to cover the wearer's crotch and a back region designed to cover the wearer's buttocks. Such products are described in U.S. Pat. No. 6,497,696 issued to Freiburger et al. and U.S. Pat. No. 6,503,239 issued to Bruemmer-Prestley et al.

An adjustable undergarment, also sometimes referred to as refastenable underwear, has a unitary configuration and can be positioned onto the wearer's body similar to regular cloth underwear. However, the adjustable undergarment has the ability to be opened and then refastened into a closed position during use. This is accomplished by having a pair of perforation or tear lines, each of which extends from the waist opening down to one of the leg openings. By tearing the product at the perforation lines, the product can be opened and inspected. If the product is still capable of absorbing and retaining additional body fluid, the product can be refastened by a pair of tabs that extend across the pair of perforation lines. The DEPEND Adjustable Underwear, manufactured and sold by Kimberly-Clark Corporation, is an example of adjustable absorbent underwear.

Most disposable absorbent incontinence underwear having an open configuration rely on some sort of an attachment system to secure the product around the wearer's torso. Such an attachment system can include a belt, a strap, a pair of straps, one or more adhesive tabs, one or more VELCRO tabs, etc. VELCRO is a registered trademark of Velcro USA, Inc., having an office at 406 Brown Avenue, Manchester, N.H. 03103. For example, each of a pair of flexible, elastic straps can be releasably secured to the back region, approximate the corners thereof, and extend forward across the wearer's hips and be releasably secured to the front region. The pair of straps will cooperate to retain the incontinence product in a comfortable fashion about the wearer's torso. When a pair of straps is utilized, the left and right hip areas are normally open and uncovered. This is different from disposable absorbent incontinence underwear having a unitary configuration where the underwear completely encircles the waist and leg openings and the front and back regions are secured together by side seams.

As stated above, disposable absorbent incontinence products are manufactured in a variety of shapes and configurations. Another type of incontinence product is a “guard for men,” which resembles an absorbent pad which can conform to the male genitalia, see U.S. Pat. No. 5,558,659 issued to Sherrod et al. The guard for men product is configured and designed to be positioned adjacent to a male wearer's penis and can be held in position by adhesive that is releasably attached to the inside surface of conventional cloth underwear. Other types of disposable absorbent incontinence products are known to those skilled in the art.

A “belted shield” is still another type of a disposable absorbent incontinence product that has an open configuration and is held about the wearer's torso by a belt or a pair of straps. A belted shield can be smaller in overall surface area such that it does not cover as much of the wearer's torso as underwear having an open configuration. Such a product is described in U.S. Pat. No. 5,386,595 issued to Kuen et al. and U.S. Pat. No. 4,886,512 issued to Damico et al.

Referring again to FIG. 1, the second segment is directed primarily to female incontinence customers, shoppers, choosers and users, herein collectively referred to as female incontinence consumers. The female incontinence consumers will be interested in various incontinence products, including pads, pantiliners, fitted briefs, belted shields, protective underwear and adjustable underwear. Some of the disposable absorbent female incontinence products in the second segment will have an absorbent core positioned in a different location on the chassis of the underwear than those in the first segment. In the female incontinence products, the absorbent core is more centered on the chassis so that it is equally spaced from the front and back waist edges. The various sizes of the disposable absorbent female incontinence underwear can have different absorbent capacities. However, like the male incontinence underwear discussed above, it is possible to design and manufacture the female incontinence underwear so that they have a single absorbent capacity or two or more absorbent capacities. Furthermore, if desired, the female incontinence underwear can be designed and manufactured to have the same absorbent capacity as the male incontinence underwear.

It should be noted that female incontinence underwear is a totally different product from pads, pantiliners, fitted briefs and belted shields, and one skilled in the art would not expect all of these products to have the same absorbent capacity as female incontinence underwear.

Disposable absorbent female incontinence underwear is available in a plurality of sizes to fit the particular rise and waist circumference of a consumer. By varying the rise and waist circumference of a product, it can better accommodate the anatomical shape of certain consumers. Female incontinence underwear is normally designed to have a specific range of rise and waist opening dimensions to comfortably fit the female consumer. Desirably, the female incontinence underwear will have a different rise and waist circumference than the male incontinence underwear.

One of the female incontinence products mentioned above is pads. By “pads” it is meant an absorbent product having an approximately rectangular, hourglass or asymmetrical configuration having a thickness of about 2.5 centimeters (cm) or less. Desirably, the thickness of a pad is less than about 10 cm. More desirably, the thickness of a pad is less than about 0.7 cm. A pad can have a length of from between about 15 cm to about 50 cm and a width of from between about 2 cm to about 15 cm. A pantiliner is another female incontinence product. By “pantiliner” it is meant a thin absorbent product having an approximately rectangular, hourglass or asymmetrical configuration having a thickness of about 1 cm or less. Desirably, the thickness of a pantiliner is less than about 0.9 cm. More desirably, the thickness of a pantiliner is less than about 0.5 cm. A pantiliner can have a length of from between about 15 cm to about 50 cm and a width of from between about 2 cm to about 15 cm. POISE is a brand name for incontinence pads and pantiliners which are manufactured by Kimberly-Clark Corporation.

Pads and pantiliners are constructed with a liquid-permeable bodyside liner, a liquid-impermeable outer cover and an absorbent enclosed therebetween. The bodyside liner is designed to contact the wearer's body when the product is worn. The bodyside liner can be made from a woven or non-woven material that will readily allow discharged body fluid or liquids to pass therethrough. The bodyside liner is normally a very thin web that can be formed from natural or synthetic fibers, with or without apertures formed therein. Spunbond and a bonded carded web are two materials that work well as a bodyside liner. “Spunbond” is a material that is manufactured and commercially sold by Kimberly-Clark Corporation.

The absorbent can be formed from natural or synthetic materials. Normally, the absorbent is made from cellulosic fibers, textile fibers, wood pulp or a combination of fibers and pulp. The absorbent can also be made from any other absorbent material known to those skilled in the art. A superabsorbent can be added to the pulp or fibers to increase the absorbent capacity of the disposable absorbent incontinence product. A superabsorbent is a material having a high capacity for absorbing and retaining a liquid or a fluid. Superabsorbents are commonly utilized in solid form and can vary in shape. Typically, small particles, granules, flakes, etc. are used. If one wishes to incorporate a superabsorbent, it can be mixed, combined, attached, printed or otherwise added to the absorbent.

The liquid-impermeable outer cover is located on the exterior of the disposable absorbent incontinence product, away from the skin of the user. The liquid-impermeable outer cover is formed from a material which will prevent or restrict fluid or liquid from penetrating or passing therethrough so as to prevent the outer clothing of the wearer from becoming soiled. Desirably, the outer cover has a soft feel so as not to chafe the inner thighs of the wearer. The outer cover can be formed from natural or synthetic fibers. The outer cover should be formed from a material that is not noisy when squeezed or wrinkled so that the disposable absorbent incontinence product remains quiet and discreet. Desirably, the outer cover is constructed from a breathable material. The outer cover can further be formed from a laminate wherein at least one layer of the laminate is liquid impervious. Examples of various materials that can be used to construct the outer cover include: a film formed from a polyolefin, such as polypropylene or polyethylene; a laminate formed from a liquid impervious layer bonded to a spunbond layer; or a laminate formed from a thermoplastic material, such as a plastic film, bonded to a spunbond layer. Other materials known to those skilled in the art can also be utilized.

The disposable absorbent incontinence pad is configured and designed to be positioned adjacent to the female's urethra and can be held in position by adhesive that is releasably attached to the inside surface of conventional cloth underwear. Alternatively, the disposable absorbent incontinence pad can be directly attached to the female wearer's skin by one or more adhesive strips.

The female incontinence pads, pantiliners, fitted briefs, belted shields, protective underwear and adjustable underwear products, included in the second segment, are similar to those male incontinence products included in the first segment except that in female incontinence underwear, the midpoint of the absorbent is normally positioned close to the middle of the crotch region. In other words, in male incontinence underwear, the absorbent is moved forward toward the front waist opening to better accommodate the male anatomy.

Still referring to FIG. 1, the third segment of incontinence products is directed primarily to consumers, shoppers, chooser and helpers, herein collectively referred to as caregivers. A “caregiver” is a person who is willing to assist another person. The person being assisted may suffer from incontinence, may be a person who is not incontinent but may have limited mobility in getting to a restroom, or is a person who is bedridden. The third segment includes at least two distinct products bearing indicia denoting targeted applicability to caregivers. A caregiver's duties can vary but can include at least one of the following activities: taking an incontinence person or a person who uses incontinent products shopping for disposable absorbent incontinence products; shopping and purchasing disposable absorbent incontinence products for another person; assisting in applying and/or removing disposable absorbent incontinence products from the body of a person who needs to wear incontinence products; talking to a person about various disposable incontinence products; and/or suggesting to a person who may use incontinent products some of the various disposable incontinence products that are available.

Anyone can serve as a caregiver. Typically, a caregiver is a family member, a relative or a person hired to provide assistance to the person in need. However, a caregiver could also be a person who only occasionally shops for or assists a person who requires assistance. For example, a son or a daughter can be a caregiver to one or both of their aging parents. Also, a grandson or a granddaughter can be a caregiver to their grandparents. A registered nurse or a nurse's aid can serve as a caregiver. The caregiver could also be a friend, a neighbor, a church member or a close acquaintance. Besides shopping and/or purchasing disposable absorbent incontinence products, a caregiver may provide suggestions to the person suffering from incontinence as to what type or brand of disposable absorbent incontinence product the person should try.

Many caregivers will shop and purchase the disposable absorbent incontinence products at a retail store, such as a grocery store, a drug store, a discount store, mass merchandiser, such as Wal-Mart or Target, or a warehouse club, such as Sam's Club or Costco. Alternatively, a caregiver may acquire the disposable absorbent incontinence products by going online to a particular web site using a computer. Still another way for a caregiver to shop and purchase disposable absorbent incontinence products is to order such incontinence products by telephone from a mail order business that will ship or mail such incontinence products directly to either the caregiver's address or to the user's address.

The caregiver may be interested in purchasing one or more of the following distinct incontinence products: pads, pantiliners, fitted briefs, guards for men, belted shields, protective underwear, adjustable underwear, as well as hygiene products, protective products and/or cleaning products required or desired by the person they are assisting. By “hygiene” it is meant the science of health and the prevention of disease. Hygiene products serve to promote and/or preserve the health of a person who suffers from incontinence, has limited mobility or is bedridden. By “protective product” it is meant any article, garment or device used to protect or prevent a person or their clothing or bedding from being soiled or contacted by a foreign substance. The foreign substance could be a liquid or a solid. For example, a liquid could include water, a drinkable fluid, such as orange juice or a soda, medicine in liquid form, etc. An example of a solid could include edible food, pills, drugs, etc. A person who is suffering from incontinence, has limited mobility or is bedridden can benefit from hygiene products because any of these conditions can lead to skin rashes, lesions and even skin disease. Hygiene products that may be of particular interest to a person who is suffering from incontinence, has limited mobility or is bedridden can include soap, lotions, creams, salves, ointments, liniments, body wipes either in dry, moist or wet form, as well as various cleaning products, especially skin cleaning products. Protective products that may be of particular interest to a person who is suffering from incontinence, has limited mobility or is bedridden can include bed pads, bibs, towels, wipes, napkins, blankets, safety glasses, liquid intake devices such as straws, and specially designed cups and lids, or solid food intake devices, such as specially designed spoons, forks, plates, trays, etc. Cleaning products can include a variety of products used to clean one or more parts of a human body. For example, hair shampoo, hair conditioners, hair spray, body lotion, body gels, body soaps, face lotions, face creams, hand soaps, hand gels, mouth wash, eye solutions, etc.

Some of the various disposable absorbent incontinence caregiver products can have different absorbent capacities. The absorbent capacity of some of the disposable absorbent incontinence caregiver products can be equal to the absorbent capacity of some of the male incontinence products or be equal to the female incontinence products. Desirably, the third segment will include at least some caregiver products that have a higher absorbent capacity than the male incontinence products in the first segment. Likewise, the third segment will include at least some caregiver products that have a higher absorbent capacity than the female incontinence products in the second segment.

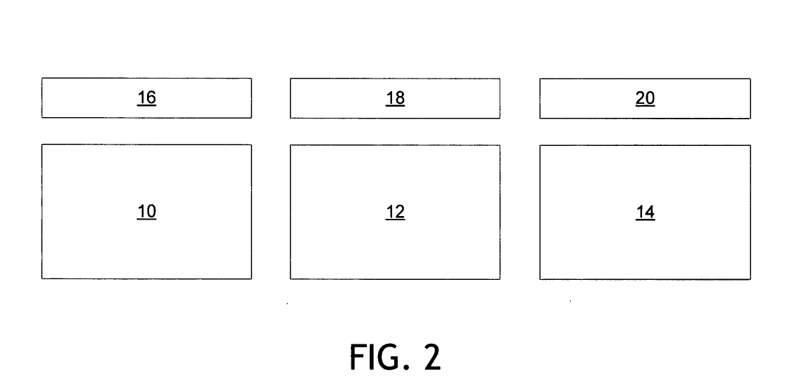

Referring now to FIG. 2, it was stated above that the method of marketing incontinence products involves distinguishing the first, second and third segments from one another by display segmentation elements. In FIG. 2, a first segment 10 is depicted represented by a box. Located adjacent to or in close proximity to the first segment 10 is a second segment 12, also represented by a box. Likewise, located adjacent to or in close proximity to the second segment 12 is a third segment 14, also represented by a box. Each box is a schematic representing a group or family of unique but different incontinence products, hygiene products, protective products or cleaning products that are specifically designed primarily for a male consumer, a female consumer or a caregiver. Each of the first, second and third segments, 10, 12 and 14 respectively, has a display segmentation element 16, 18 and 20 respectively, associated with it. In FIG. 2, a first display segmentation element 16 is associated with the first segment 10, a second display segmentation element 18 is associated with the second segment 12, and a third display segmentation element 20 is associated with the third segment 14. By “associated” it is meant that the display segmentation element 16, 18 or 20 is directly or indirectly attached, secured, joined or positioned relative to the respective segment. For example, the display segmentation element 16, 18 or 20 can be positioned in close proximity to the respective segment 10, 12 or 14 respectively. Alternatively, the display segmentation element 16, 18 or 20 can be the actual packaging that encloses at least one of the incontinence, hygiene, protective and/or cleaning products situated within a particular segment.

By a “display segmentation element” it is meant a package, a sign, a display, one or more words, a phrase, distinctive graphics on a package, a graphical image, a particular color, an icon or some other kind of indicator directing the consumers attention to a specific area. For example, a display segmentation element can be a uniquely shaped package or a package bearing a unique design that is recognizable by the ultimate consumer. A display segmentation element can also be distinctive graphics on a package which includes indicia denoting targeted applicability to male incontinence consumers, female incontinence consumers or to caregivers. The brand name on the package, the package graphics, the package background color, etc. can all serve the function of being a display segmentation element for they assist the consumer in identifying the correct product they wish to buy. Another example of a display segmentation element is a sign, an icon, an image, such as a graphical image, letters, words, phrases, etc. located above, adjacent to or in close proximity to the male incontinence products, to the female incontinence products and to the caregiver products. The sign or image will assist in focusing the consumer's attention on the kind and type of incontinence products they wish to purchase. Other examples of a display segmentation element include but are not limited to a particular retail aisle, a retail shelf, an end of an aisle display or an area on a retail shelf. On a computer, a display segmentation element can be a particular web site, a web page, an icon, a drop down menu or any other image or graphical display known to those skilled in the art for directing a potential purchaser to a specific product.

It should also be noted that the display segmentation element can direct the consumer's attention to hygiene, protective and/or cleaning products, etc. that are also present in at least one of the first, second and third segments 10, 12 and 14 respectively. Such hygiene, protective and/or cleaning products, etc. can be specifically designed for a male consumer, for a female consumer or for a caregiver.

Referring again to FIG. 1, the time has come to change the way incontinence products, especially disposable absorbent incontinence products, are marketed to the consumers. The method is designed to make it easier for male and female incontinence consumers, as well as caregivers, to locate, identify and select the particular product that they wish to purchase or that meet their incontinence or hygiene needs. The method described herein includes communicating the introduction or availability of a particular incontinence product, hygiene product, protective product, cleaning product or an array of such products, to consumers using multimedia advertisements. By “multimedia” it is meant using or involving the use of several media. Multimedia advertisements include but are not limited to oral, written and graphical advertisements. Examples of oral advertisements includes a personal discussion, a group discussion, a lecture, telephone advertisement, radio advertisement, television advertisement and voice advertisement using a computer with a sound card. These and other kinds and types of oral advertisement, known to those skilled in the art, can be used.

Examples of written advertisements include hand written advertisement, a typed advertisement, a printed advertisement, an electronically generated advertisement or a computer generated advertisement. These and other kinds and types of written advertisement, known to those skilled in the art, can be used. Lastly, examples of graphical advertisements include but are not limited to pictorial representations, pictures, charts, drawings, graphs, symbols, visual art, computer displayed graphics, engravings, lithographs, electronic images and computer images. These and other kinds and types of graphical advertisement, known to those skilled in the art, can be used.

The method of marketing further includes creating a product display at a retail site that resembles the product offering communicated to the consumers. By “retail site” it is meant a typical retail outlet or a virtual outlet. A typical retail outlet includes a grocery store, a drug store, a supermarket, a discount store, a mass merchandiser, such as Wal-Mart or Target, a warehouse club, such as Sam's Club or Costco, or any other kind of brick and mortar store, outlet or place where merchandise is offered for sale. By “virtual outlet” it is meant a virtual store which can be accessed on-line using a computer, such as a specific website.

The method of marketing incontinence products, especially disposable absorbent incontinence products, is especially adapted to marketing a predominantly gender specific platform of products. By a “gender specific platform of products” it is meant that a group or family of incontinence, hygiene, protective or cleaning products are manufactured to male or female users. Another group or family of incontinence, hygiene, protective or cleaning products can be manufactured specifically for female users. Still another group or family of incontinence, hygiene, protective or cleaning products can be manufactured specifically for caregivers.

The male incontinence products can include at least two sizes of underwear, for example a small/medium size and a large size. The underwear can be constructed such that the absorbent core has the same absorbent capacity in each of the two sizes so that each product is capable of absorbing the same amount of body fluid. This means that a single size absorbent core, having a predetermined length, width, thickness and composition, can be manufactured and be inserted into each of the two different sized underwear. By doing this, the manufacturer can reduce his overall cost and possibly speed up his production process. Alternatively, all of the male incontinent underwear and some of the female incontinent underwear can be manufactured with a single absorbent capacity. For example, a small/medium-size male incontinent underwear can be manufactured to have a chassis length of about 800 mm and an absorbent core, which is secured to the chassis, having a length of about 500 mm. In the large-size male incontinent underwear, the chassis can be made longer, such as to a length of about 850 mm or more while the length of the absorbent core remains the same as in the small/medium-size male incontinent underwear. One or more of the female incontinent underwear can be manufactured to match up with the absorbent capacity of one of the male incontinent underwear. It should be noted that in this scenario, the small/medium-size male and female incontinent underwear may contain a greater absorbent capacity than needed but the cost advantage of producing two or more sizes of male and or female incontinent underwear with an identical absorbent core will more than offset the cost of the extra absorbent fiber or pulp present in the small/medium-size incontinent underwear.

This synergy can be taken a step further wherein two or more sizes of the female incontinent underwear can be constructed so as to utilize the same size absorbent core having the same absorbent capacity as two or more sizes of male incontinent underwear. The only difference is that in the male incontinent underwear, the absorbent core may be shifted forward so that it is aligned closer to the front waist opening. In the female incontinent underwear, the absorbent core is shifted further back so that it is nearer the center of the crotch region. This positioning of the absorbent core in the different incontinence underwear will allow the absorbent core to better line up with the urethral openings of the male and female users. Hence, the functionality of the disposable absorbent incontinence underwear is increased. Absorbent cores tailored to a specific gender require less absorbent material because the absorbent core can be specifically placed in the region of the male penis or the female urethral opening. Hence, the absorbent core can be shorter in length than for unisex absorbent products. This positioning of more uniquely sized absorbent cores allow for more comfortable products and results in substantial cost savings because less absorbent material is required.

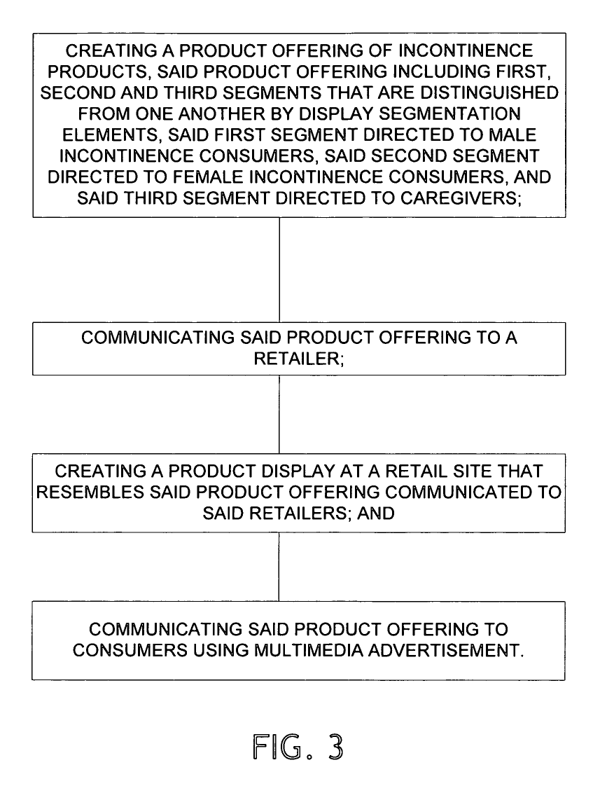

Referring now to FIG. 3, a second flow diagram is shown of an alternative embodiment of marketing incontinence products. This method differs from that depicted in FIG. 1 in that a product offering is first communicated to at least one retailer. The manufacturer can suggest a “product display” to the retailer which matches his product offering. By “product display” it is meant an arrangement of specific products relative to one another. For example, a fast moving product could be positioned at eye level to the ultimate consumers while a slower moving product could be placed lower within the display. The retailer can then create a product display at his retail site that resembles the product offering communicated to him. The product offering is then communicated to the consumer. One benefit of marketing incontinence products this way is that the manufacturer may be better able to control exactly what is conveyed to both parties. Hopefully, the same message is being communicated to each. For example, a manufacturer having a new lineup of male, female and caregiver incontinence, hygiene, protective and/or cleaning products, can use multimedia advertisements, i.e. television and print advertisements, to communicate the new products to the consumers either simultaneously or sequentially after communicating a product display to his retailers. In doing so, the manufacturer can suggest to the retailer what products should be stocked and sold and how the products should be displayed. Assuming that the retailer agrees to display the products as suggested by the manufacturer, subsequent advertisements can be disseminated to the consumer notifying them of where and when the products will be available for purchase. By first communicating product introductions and product displays to the retailer, the manufacturer can avoid confusion for the consumers when they enter the retailer's establishment to purchase their products.

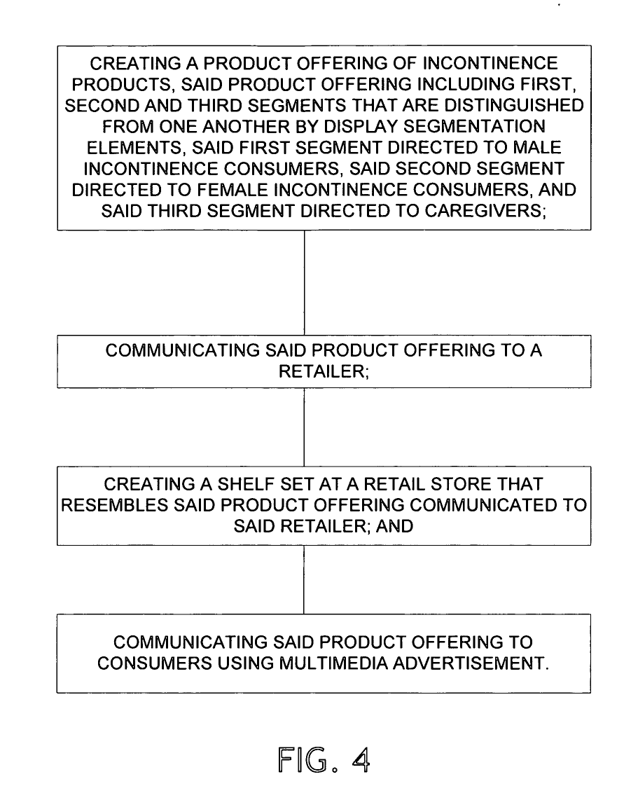

Referring now to FIG. 4, a third flow diagram is shown of still another method of marketing incontinence products. This method differs from that depicted in FIG. 2 in that the manufacturer can suggest to a retailer a particular “shelf set” to use. By “shelf set” it is meant the arrangement and layout of the male, female and caregiver incontinence, hygiene, protective and/or cleaning products on the store shelf. If the retailer agrees to use the particular shelf set suggested by the manufacturer, this information can be communicated back to the ultimate consumer in additional or subsequent advertisements so that they will know exactly where their particular incontinence, hygiene, protective and/or cleaning products are located on the store shelf. This method further avoids confusion at the retail store by having the consumer know exactly where to look on the store shelf for his or her favorite incontinence, hygiene, protective and/or cleaning product.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A method of marketing incontinence products, comprising:
a) creating a product offering of incontinence products, said product offering including first, second and third segments that are distinguished from one another by display segmentation elements, said first segment directed to male incontinence consumers, said second segment directed to female incontinence consumers, and said third segment directed to caregivers;
b) communicating said product offering to consumers using multimedia advertisements; and
c) creating a product display at a retail site that resembles said product offering communicated to said consumers.

2. The method of claim 1 wherein said multimedia advertisement includes oral, written and graphical advertisements.

3. The method of claim 2 wherein said oral advertisement include at least one of the following: a personal discussion, a group discussion, a lecture, a telephone advertisement, a radio advertisement, a voice advertisement using a computer and a television advertisement.

4. The method of claim 2 wherein said written advertisement includes at least one of the following: a hand written advertisement, a typed advertisement, a printed advertisement, an electronically generated advertisement and a computer generated advertisement.

5. The method of claim 2 wherein said graphical advertisements include at least one of the following: a pictorial representation, a picture, a chart, a drawing, a graph, a symbol, visual art, a computer displayed graphic, an engraving, a lithograph, an electronic image and a computer image.

6. The method of claim 1 wherein said display segmentation elements are signs.

7. The method of claim 1 wherein said display segmentation elements are graphical images.

8. The method of claim 1 wherein said first segment includes at least two distinct products bearing indicia denoting targeted applicability to male consumers.

9. The method of claim 1 wherein said third segment includes adjustable underwear.

10. A method of marketing incontinence products, comprising:
a) creating a product offering of incontinence products, said product offering including first, second and third segments that are distinguished from one another by display segmentation elements, said first segment directed to male incontinence consumers, said second segment directed to female incontinence consumers, and said third segment directed to caregivers;
b) communicating said product offering to a retailer;
c) creating a product display at a retail site that resembles said product offering communicated to said retailer; and
d) communicating said product offering to consumers using multimedia advertisement.

11. The method of claim 10 wherein said first and second segments include various products each having a chassis and an absorbent core, and some of said products in said second segment have said absorbent core positioned in a different location on said chassis than said products in said first segment.

12. The method of claim 10 wherein said third segment includes at least two distinct products bearing indicia denoting targeted applicability to caregivers, and said distinct products include at least six of the following: pads, fitted briefs, protective underwear, adjustable underwear; hygiene products such as soaps, lotions, creams, salves, ointments, liniments; cleaning products such as wet wipes, dry wipes and moist wipes; and protective products such as bed pads, bibs, napkins and towels.

13. The method of claim 10 wherein said display segmentation elements include: a sign, a display, a product package, a distinctive graphic on a package, a background color on a package, a retail aisle, a retail shelf or an area on a retail shelf.

14. The method of claim 10 wherein one of said display segmentation elements can be a distinctive graphic on a package which includes indicia denoting targeted applicability to one of said first, second and third segments.

15. A method of marketing disposable incontinence products, comprising:
a) creating a product offering of incontinence products, said product offering including first, second and third segments that are distinguished from one another by display segmentation elements, said first segment directed to male incontinence consumers, said second segment directed to female incontinence consumers, and said third segment directed to caregivers;
b) communicating said product offering to a retailer;
c) creating a shelf set at a retail store that resembles said product offering communicated to said retailer; and
d) communicating said product offering to consumers using multimedia advertisement.

16. The method of claim 15 wherein said first segment includes various sizes of male underwear all having a single absorbent capacity.

17. The method of claim 15 wherein said second segment includes various sizes of female underwear all having a single absorbent capacity.

18. The method of claim 15 wherein said third segment includes at least two distinct product forms bearing indicia denoting targeted applicability to caregivers, and said distinct products include at least six of the following: pads, fitted briefs, protective underwear, adjustable underwear; hygiene products such as soaps, lotions, creams, salves, ointments, liniments; cleaning products such as wet wipes, dry wipes and moist wipes; and protective products such as bed pads, bibs, napkins and towels.

19. The method of claim 15 wherein said third segment includes at least some products that have a higher absorbent capacity than products in said first segment.

20. The method of claim 15 wherein said first and second segments include various underwear each having a rise and a waist circumference, and said underwear in said first segment have a rise and waist circumference different from said rise and waist circumference of said underwear in said second segment.
